(19) United States
(12) Patent Application Publication
(10) Pub. No.: US 2018/0296171 A1
(43) Pub. Date: Oct. 18, 2018

(54) RADIATION IMAGING APPARATUS, RADIATION IMAGING METHOD, CT APPARATUS, AND STORAGE MEDIUM

(71) Applicant: CANON KABUSHIKI KAISHA, Tokyo (JP)

(72) Inventor: Jumpei Shirono, Yokohama-shi (JP)

(21) Appl. No.: 16/018,155

(22) Filed: Jun. 26, 2018

Related U.S. Application Data
(63) Continuation of application No. PCT/JP2016/080831, filed on Oct. 18, 2016.

(30) Foreign Application Priority Data
Dec. 28, 2015 (JP) 2015-257322

(51) Int. Cl. A61B 6/03; A61B 6/00

(52) U.S. Cl. CPC A61B 6/032; A61B 6/482; G01N 2223/206; A61B 6/542; A61B 6/52; A61B 6/4241

(57) ABSTRACT

A radiation imaging apparatus includes: a detection unit configured to obtain measurement information based on a detection result of radiation irradiated based on a constant tube voltage; an obtaining unit configured to obtain second measurement information of the radiation based on a moment of the measurement information obtained by detecting the radiation multiple times; an energy determination unit configured to determine a plurality of energies for approximating an energy distribution of the radiation; and a calculation unit configured to calculate photon counts corresponding to the plurality of energies based on the second measurement information.

### CROSS-REFERENCE TO RELATED APPLICATIONS

This application is a Continuation of International Patent Application No. PCT/JP2016/080831, filed Oct. 18, 2016, which claims the benefit of Japanese Patent Application No. 2015-257322, filed Dec. 28, 2015, both of which are hereby incorporated by reference herein in their entirety.

### BACKGROUND OF THE INVENTION

### Field of the Invention

The present invention relates to a radiation imaging apparatus, a radiation imaging method, a CT apparatus, and a storage medium.

### Background Art

A radiation imaging apparatus is an apparatus that visualizes attenuation of radiation that has transmitted through an object as lightness and darkness of pixels (a grayscale image), based on the radiation intensity (energy) detected by a detection apparatus. Portions inside of the object (e.g., bone, fat, muscle, etc.) have different radiation transmittances, and therefore, for example, at a portion that has little radiation absorption, the radiation intensity that reaches the detection apparatus is high, and at a portion that has high radiation absorption, the radiation intensity that reaches the detection apparatus is low. Thus, the level of attenuation of the radiation differs depending on which portion inside of the object is transmitted through. With a conventional radiation imaging apparatus, a grayscale image is generated based on the attenuation of the radiation that has transmitted through the object, but if the levels of attenuation of the radiation are the same, the information of the portions inside of the object cannot be obtained as a grayscale image.

PTL 1 discloses a technique of performing multiple instances of radiation imaging at different tube voltages of a radiation generation unit, whereby average photon counts corresponding to the energies of the radiation that was irradiated at the tube voltages is obtained, and thus the portions inside of the object are estimated.

However, with the configuration disclosed in PTL 1, an operator needs to change the tube voltage in order to irradiate the radiation, and if a motion artifact is generated due to the object moving while the tube voltage is being switched, the measurement accuracy will decrease, and therefore the number of photons cannot be calculated with high accuracy based on the measurement result.

In view of the foregoing circumstance, the present invention provides a radiation imaging technique that can obtain multiple pieces of energy information of radiation irradiated based on a constant tube voltage, and can calculate with high precision the photon counts corresponding to the pieces of energy information, without being influenced by a decrease in the measurement accuracy.

### CITATION LIST

### Patent Literature

PTL1: Japanese Patent Laid-Open No. 2009-285356

### SUMMARY OF THE INVENTION

A radiation imaging apparatus according to an aspect of the present invention includes: a detection unit configured to obtain measurement information based on a detection result of radiation irradiated based on a constant tube voltage; an obtaining unit configured to obtain second measurement information of the radiation based on a moment of the measurement information obtained by detecting the radiation a plurality of times; an energy determination unit configured to determine a plurality of energies for approximating an energy distribution of the radiation; and a calculation unit configured to calculate photon counts corresponding to the plurality of energies based on the second measurement information.

A radiation imaging apparatus according to another aspect of the present invention includes: a detection unit configured to obtain measurement information based on a detection result of radiation irradiated based on a constant tube voltage; an obtaining unit configured to obtain second measurement information of the radiation based on a moment of the measurement information obtained by detecting the radiation a plurality of times; an energy determination unit configured to determine a plurality of energies for approximating an energy distribution of the radiation; a calculation unit configured to calculate photon counts corresponding to the plurality of energies based on the second measurement information; and a display control unit configured to display an image based on the photon counts on a display unit.

Further features of the present invention will become apparent from the following description of exemplary embodiments with reference to the attached drawings.

### BRIEF DESCRIPTION OF THE DRAWINGS

FIG. 1 is a diagram illustrating a configuration of a radiation imaging apparatus according to an embodiment.

FIG. 2 is a diagram illustrating a processing flow for calculating an average photon count.

FIG. 3 is a diagram in which radiation that is emitted from a radiation generating apparatus passes through an object and is incident on a detection element.

FIG. 4 is a diagram illustrating a configuration of a CT apparatus according to an embodiment.

FIG. 5 is a diagram illustrating a processing flow for calculating a linear attenuation coefficient.

FIG. 6 is a diagram showing an example of a two-dimensional image based on an integrated value of energy of radiation.

FIG. 7 is a diagram illustrating a result of obtaining average photon counts.

FIG. 8 is a diagram illustrating an image based on a radiation photon count distribution.

FIG. 9 is a diagram illustrating a configuration of a radiation imaging apparatus according to an embodiment.

FIG. 10 is a diagram illustrating a configuration of a CT apparatus according to an embodiment.

### DESCRIPTION OF THE EMBODIMENTS

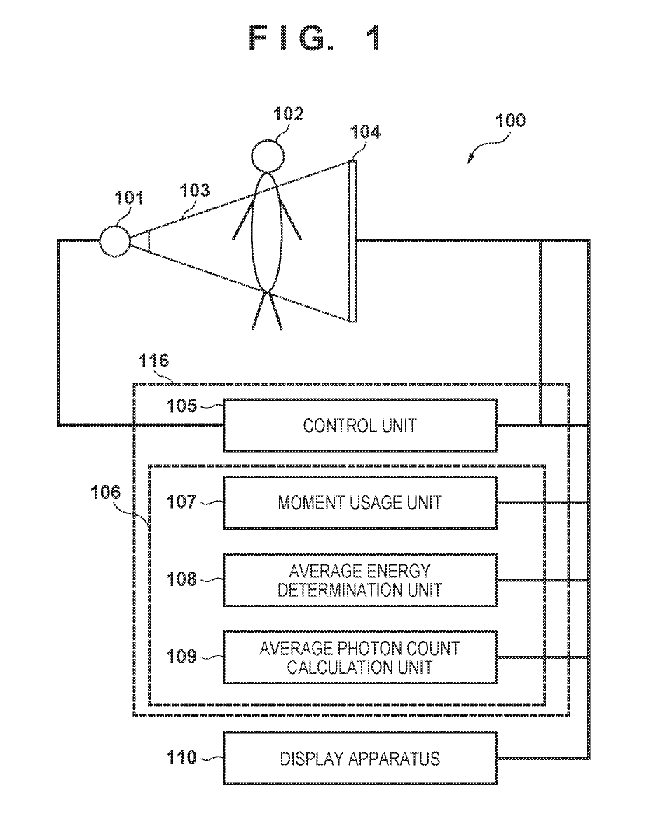

Hereinafter, embodiments of the present invention will be described illustratively and in detail with reference to FIGS. 1 to 10. Note that the constituent elements described in the embodiment are merely examples, the technical scope of the present invention is established by the claims, and there is no limitation to the following individual embodiments.

FIG. 1 is a diagram showing an example of a configuration of a radiation imaging apparatus 100 of an embodiment. As shown in FIG. 1, the radiation imaging apparatus 100 includes a radiation generating apparatus 101, a radiation detection apparatus 104, and an information processing apparatus 116. Note that this configuration is also called a radiation imaging system. The information processing apparatus 116 includes a control unit 105 that controls the operations of the radiation generating apparatus 101 that irradiates radiation and the radiation detection apparatus 104, and a data processing unit 106 (image processing unit) that processes data detected by the radiation detection apparatus 104. Also, for example, a display apparatus 110 constituted by a liquid crystal display, a CRT, or the like is connected to the information processing apparatus 116, and the display apparatus 110 displays the processing result of the data processing unit 106. The control unit 105 functions also as a display control unit that controls display of the display apparatus 110.

The control unit 105 functions as a mechanism control unit to perform position control of the radiation generating apparatus 101 and the radiation detection apparatus 104. Also, the control unit 105 functions as an irradiation control unit to cause the radiation generating apparatus 101 to irradiate radiation based on a constant tube voltage. That is, the control unit 105 performs control to apply a set constant tube voltage to the radiation generating apparatus 101, and thus controls the irradiation of the radiation performed by the radiation generating apparatus 101. The radiation generating apparatus 101 outputs the radiation based on the control performed by the control unit 105. Reference numeral 103 schematically indicates the radiation emitted from the radiation generating apparatus 101. The radiation is X rays, α rays, β rays, or γ rays, for example.

The control unit 105 functions as an imaging control unit to control the operations of the radiation generating apparatus 101 and the radiation detection apparatus 104, thereby causing multiple instances of radiation imaging to be executed in a predetermined amount of time and causing detection data (measurement information) to be output from the radiation detection apparatus 104. The control unit 105 causes the radiation to be irradiated from the radiation generating apparatus 101 based on a constant tube voltage and controls the radiation detection apparatus 104 to output the detection results of the radiation incident on the detection units of the radiation detection apparatus 104 each certain period, and thus obtains the measurement information. For example, the control unit 105 controls the radiation generating apparatus 101 so as to irradiate radiation at a constant tube voltage, and can cause the detection result of the radiation incident on the detection units of the radiation detection apparatus 104 to be output as detection data (measurement information) each certain period.

A detection unit of the radiation detection apparatus 104 outputs measurement information that is proportional to the sum of the energies of the radiation that is incident for a certain time period (e.g., a predetermined time period (one frame)). The radiation detection apparatus 104 can obtain measurement information based on the detection result of the radiation irradiated based on the constant tube voltage. Specifically, the radiation detection apparatus 104 includes a detection unit (detection element) that detects radiation that was irradiated based on the constant tube voltage, and the detection unit outputs the total energy (integrated value) of the radiation incident on the detection unit for every certain time period (1 frame) as detection data (measurement information). For example, the radiation detection apparatus 104 includes multiple detection units (detection elements) that are arranged in a two-dimensional shape. A flat panel detector (FPD), which is constituted by a semiconductor material and in which multiple detection elements are arranged side by side in a grid shape can be used as the configuration of the radiation detection apparatus 104, and a configuration such as a line sensor can also be used thereas. It is also possible to include only one detection unit (detection element).

The radiation detection apparatus 104 uses the detection units (detection elements) to detect the intensities (energies) of the radiation that was output from the radiation generating apparatus 101 and has transmitted through the object 102. Although the object 102 is a living body in the present embodiment, it is also possible to use an object that is not a living body, such as an industrial product. If the radiation detection apparatus 104 includes a configuration for a flat panel detector, the detection units (detection elements) are arrayed in two dimensions so as to form multiple rows and multiple columns, for example. The radiation detection apparatus 104 includes a drive unit that drives the multiple detection units in units of rows or in units of columns, and the control unit 105 controls the drive unit to cause the multiple detection units (detection elements) to sequentially output the detection data (measurement information) corresponding to the total energy (integrated value) of the incident radiation.

The information detected by the detection units of the radiation detection apparatus 104 is sent to the data processing unit 106 (image processing unit) of the information processing apparatus 116 and processed. The data processing unit 106 (image processing unit) includes a moment usage unit 107 (obtaining unit), an average energy determination unit 108, and an average photon count calculation unit 109. The functions of the units of the control unit 105 and the data processing unit 106 are configured using a program read from a CPU, a GPU, or a memory (not shown), for example. The configurations of the units of the control unit 105 and the data processing unit 106 may be constituted by an integrated circuit, as long as similar functions are achieved.

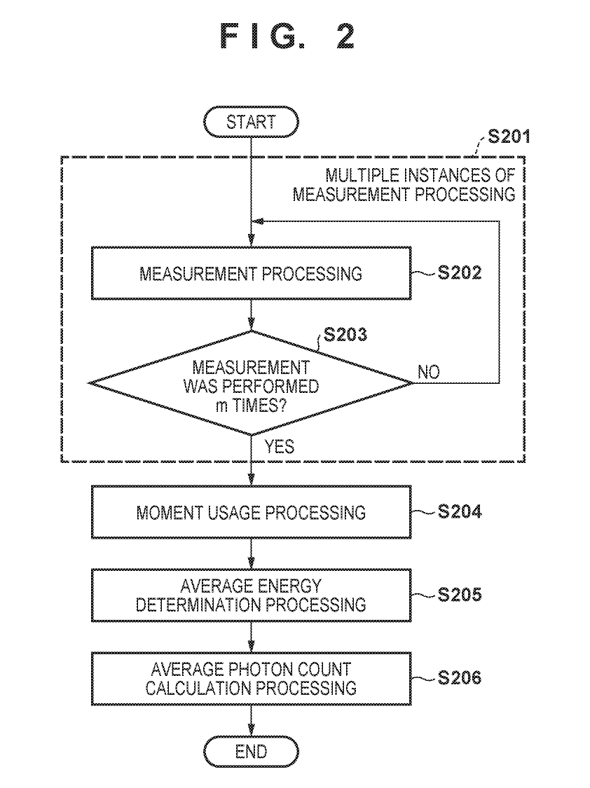

FIG. 2 is a diagram illustrating a flow of processing for calculating the average photon counts, performed by the radiation imaging apparatus 100. The operations performed by the units of the control unit 105 and the data processing unit 106 shown in FIG. 1 to calculate the average photon counts will be described with reference to FIG. 2.

### Multiple Instances of Measurement Processing (Step S201)

First, in step S201, the control unit 105 executes multiple instances of measurement processing. The control unit 105 causes the radiation generating apparatus 101 and the radiation detection apparatus 104 to operate in conjunction with each other so as to execute the multiple instances of measurement processing. The multiple instances of measurement processing include two steps, and the measurement is performed in step S202. The control unit 105 controls the radiation generating apparatus 101 so as to irradiate radiation at a constant tube voltage, and causes the detection results of the radiation incident on the detection units (detection elements) of the radiation detection apparatus 104 to be output each certain period. The measurement information measured by the detection units (detection elements) of the radiation detection apparatus 104 is denoted as dᵢ. The affix i indicates information of the measurement executed for the i-th time.

In step S203, the control unit 105 determines whether or not a predetermined number of instances (m: an integer that is 2 or more) of measurement have ended. If the predetermined number of instances (m instances) have not ended (step S203—No), the processing is returned to step S201, and the measurement is performed once again. On the other hand, in the determination of step S203, if the predetermined number of instances (m instances) of measurement have ended (step S203—Yes), the processing is advanced to step S204. By executing the predetermined number of instances (m instances) of measurement, m instances' worth of measurement information is input to the moment usage unit 107.

### Moment Usage Processing: Step S204

In step S204, the moment usage unit 107 (obtaining unit) obtains second measurement information of the radiation based on the moment of the measurement information obtained by detecting the radiation multiple times. The second measurement information includes information obtained using Equations 1 and 2 below, for example. For example, the moment usage unit 107 (obtaining unit) obtains, as the second measurement information, the average photon count (<n>) of the radiation incident on the detection units based on the moment of the measurement information dᵢ, obtained by detecting the radiation multiple times. The moment usage unit 107 uses Equation 1 to obtain the average photon count <n> as the second measurement information.

$${E}_{mean} = \frac{1}{α} \frac{〈 {( d - 〈 d 〉 )}^{2} 〉}{〈 d 〉} , 〈 n 〉 = \frac{〈 d 〉}{α {E}_{mean}} \qquad Equation 1$$

Here, a is the conversion coefficient of the measurement information and the average energy, and Eₘₑₐₙ is the average energy. The method for determining the conversion coefficient α is performed as follows, for example. Based on the control performed by the control unit 105, first, the radiation emitted from a radiation source (radiation generating apparatus) having a known spectral distribution is measured such that only one photon is incident on a detection unit (detection element) by weakening the intensity of the radiation in a state with no object. This measurement is implemented multiple times, and the average of the measurement information is divided by the average energy of the spectral distribution, whereby conversion coefficient α can be obtained.

<d> is a first moment about the origin, and <(d−<d>)²> is a second central moment. The moment usage unit 107 (obtaining unit) can obtain the first moment about the origin (<d>) and the second central moment (<(d−<d>)²>) through calculation using Equation 2 below.

$$〈 d 〉 = \frac{1}{m} {\sum }_{i = 1}^{m} {d}_{i} , 〈 {( d - 〈 d 〉 )}^{2} 〉 = \frac{1}{m} {\sum }_{i = 1}^{m} {( {d}_{i} - 〈 d 〉 )}^{2} \qquad Equation 2$$

The reason why the average photon count <n> can be obtained using these equations will be described in detail below. Although the photons incident on the detection units commonly have a spectral distribution, if it is assumed that approximation can be performed using the average energy Eₘₑₐₙ, the pieces of measurement information dᵢ can be written as shown in Equation 3.

dᵢ=αEₘₑₐₙn₁ Equation 3

In Equation 3, nᵢ is the photon count. The photon count nᵢ commonly has a fluctuation that follows a Poisson distribution, and it is known that in a Poisson distribution, the first moment about the origin and the second central moment are equal. That is, if the relationship between the first moment about the origin and the second central moment is expressed using the photon count nᵢ, Equation 4 below is achieved.

[symbol]

n [symbol] = [symbol] (n− [symbol] n [symbol] )²

[symbol]

If both sides of Equation 4 are multiplied by the square of αEₘₑₐₙ and adjustment is performed using Equation 3, Equations 1 and 2 can be obtained and the average photon count <n> can be obtained using Equations 1 and 2. Note that when the second central moment (Equation 2) is to be obtained, the moment usage unit 107 may divide by m−1 instead of m (number of instances of measurement), that is, the moment usage unit 107 (obtaining unit) may obtain an unbiased variance.

### Average Energy Determination Processing: Step S206

Next, in step S205, the average energy determination unit 108 (energy determination unit) determines multiple energies (average energies) for approximating an energy distribution of the radiation. The average energy determination unit 108 determines the multiple average energies based on the energy properties of the radiation irradiated from the radiation generating apparatus 101. In the present embodiment, the average energy determination unit 108 determines two average energies E₁ and E₂ as the multiple energies. Although any method can be used to determine the average energies, it is possible to set them using the spectrum of the radiation emitted from the radiation generating apparatus 101 or the energy dependency of the linear attenuation coefficient of the substance constituting the object.

In one example of a method for determining the energy in the average energy determination unit 108, the spectral distribution of the radiation is divided into multiple regions, and the average values of the energy based on the spectral distributions of the divided regions can be determined as the energies (average energies) for approximating the energy distribution of the radiation. For example, there is a method in which the spectrum of the radiation emitted from the radiation generating apparatus 101 is divided into two energy regions such that the integrated values of the spectra are equal, and the average energy for each region is determined as the average energy. That is, if the spectrum of the radiation emitted from the radiation generating apparatus 101 is g(E), the average energy determination unit 108 can set E_{d}, which satisfies Equation 5, such that the integrated values of the spectra in both regions are equal, and can determine the average energies E₁ and E₂ as in Equation 6.

$$\begin{matrix}{\int }_{- ∞}^{{E}_{d}} g ( E ) dE = {\int }_{{E}_{d}}^{∞} g ( E ) dE & Equation 5 \\ {E}_{1} = \frac{{\int }_{- ∞}^{{E}_{d}} Eg ( E ) dE}{{\int }_{- ∞}^{{E}_{d}} g ( E ) dE} , {E}_{2} = \frac{{\int }_{{E}_{d}}^{∞} Eg ( E ) dE}{{\int }_{{E}_{d}}^{∞} g ( E ) dE} & Equation 6\end{matrix}$$

Alternatively, the average energy determination unit 108 divides the spectral distribution of the radiation into multiple regions based on the energies of the absorption edges of the substances constituting the object, and thus can determine the average energies. For example, in the case of using a radiopaque dye such as iodine, the average energy determination unit 108 divides the spectrum by the energy of the absorption edge of the iodine, and the average energy of each region can be determined as the average energies. Furthermore, examples of average energy determination also include a method in which an operator designates the average energies via an input apparatus, according to experiential learning.

### Average Photon Count Calculation Processing: Step S206

Next, in step S206, the average photon count calculation unit 109 can calculate the photon count (average photon count) corresponding to each of the multiple energies (average energies) based on the second measurement information. In the present embodiment, an example will be described in which the average photon counts <n₁> and <n₂> corresponding to the average energies E₁ and E₂ are obtained based on Equation 7. The average photon count calculation unit 109 (calculation unit) calculates the photon counts corresponding to the multiple energies using the multiple energies (multiple average energies) and the second measurement information (<n>).

$$〈 {n}_{1} 〉 = \frac{〈 n 〉 {E}_{2} - 〈 d 〉 / α}{{E}_{2} - {E}_{1}} , 〈 {n}_{2} 〉 = \frac{〈 n 〉 {E}_{1} - 〈 d 〉 / α}{{E}_{1} - {E}_{2}} \qquad Equation 7$$

The reason why <n₁> and <n₂> can be obtained using these equations will be described in detail below. In general, the radiation that is incident on the detection units of the radiation detection apparatus 104 has a spectral distribution, and this spectral distribution can be approximated using the average energies E₁ and E₂. When the average photon counts corresponding to average energies E₁ and E₂ are set as n₁ and n₂, the photon count <n> incident on the detection units is constituted by the average photon counts n₁ and n₂ corresponding to the average energies E₁ and E₂, and therefore the relationship shown in Equation 8 is established.

[symbol]

n [symbol] = [symbol] n₁ [symbol] + [symbol] n₂ [symbol] Equation 8

Also, the detection unit of the radiation detection apparatus 104 outputs measurement information dᵢ that is proportional to the sum of the energies of the radiation that is incident in a certain time period (e.g., a predetermined time period (one frame)). For this reason, if the energies of the radiation can be approximated using the two average energies E₁ and E₂, they can be written as in Equation 9.

[symbol]

d [symbol] =α( [symbol] n₁ [symbol] E₁+ [symbol] n₂ [symbol] E₂) Equation 9

In Equations 8 and 9, <n₁> and <n₂> are the only unknown numbers, and therefore if a simultaneous linear equation therefor is solved, the relationship shown in Equation 7 can be derived. Accordingly, the average photon count calculation unit 109 can obtain the average photon counts <n₁> and <n₂> according to Equation 7. The control unit 105 functions also as a display control unit that controls display of the display apparatus 110, and can display, on the display apparatus 110, the average photon counts <n₁> and <n₂> obtained using the processing of the average photon count calculation unit 109, or an image based on the average photon counts <n₁> and <n₂>, as a diagnostic image.

FIG. 6 is a diagram showing an example of a two-dimensional image (integral image of radiation energy) based on the measurement information (the integrated value (Σdᵢ) of the energies of the radiation) measured by the detection units of the radiation detection apparatus (FPD). The radiation energy image is an image obtained through normal radiation imaging, and FIG. 6 shows an example in which a substance 1 (601) (e.g., bone) exists in a substance 2 (602) (e.g., soft tissue). In the integral image of the radiation energy, depending on the thickness of the substance, the integrals of the radiation energies of the substance 1 (601) and the substance 2 (602) are about the same, and therefore it is not possible to distinguish between the two substances based on the integrated values of the radiation energies. That is, in the two-dimensional image (integral image of the radiation energy), the substance 1 (601) in the substance 2 (602) cannot be distinguished.

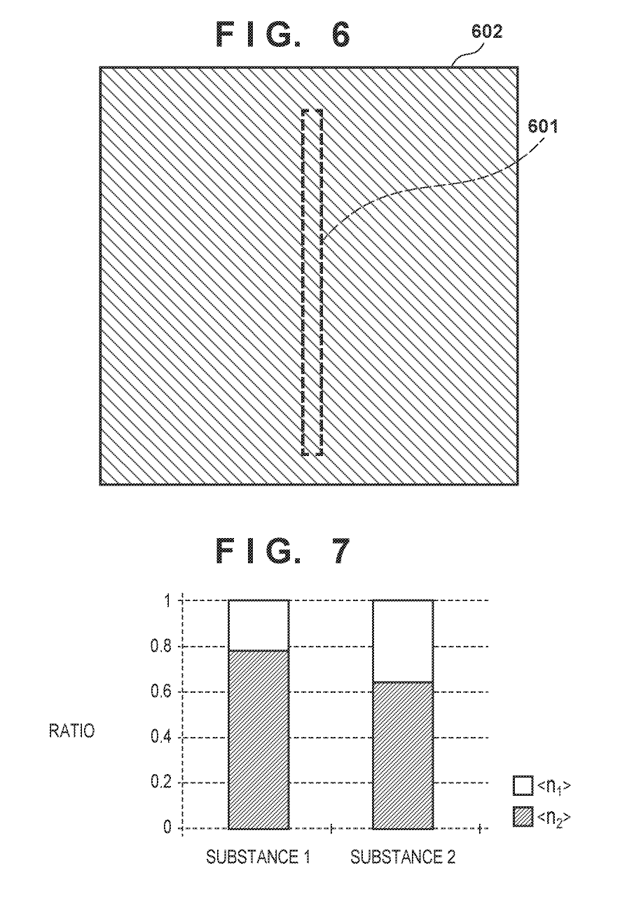

FIG. 7 is a diagram illustrating ratios of the average photon counts of the substances, by applying the processing of the present embodiment and obtaining the average photon counts <n₁> and <n₂> in the example shown in FIG. 6. Even if the integrated values of the energies of the radiation are the same values, bone contributes to beam hardening more than the soft tissue, and therefore has a higher percentage of high-energy photons. Accordingly, if the energies are selected as in Equations 5 and 6, and the processing of the present embodiment is applied to obtain the average photon counts <n₁> and <n₂> and obtain the ratio of <n₁> and <n₂>, then, for example, as shown in FIG. 7, the substance 1 (601) (bone) will have a larger ratio of a high-energy photon count (<n₂>) than the substance 2 (602) (soft tissue). Thus, even if the integrated values are the same values, it is possible to discriminate between the substances by applying the processing of the present embodiment.

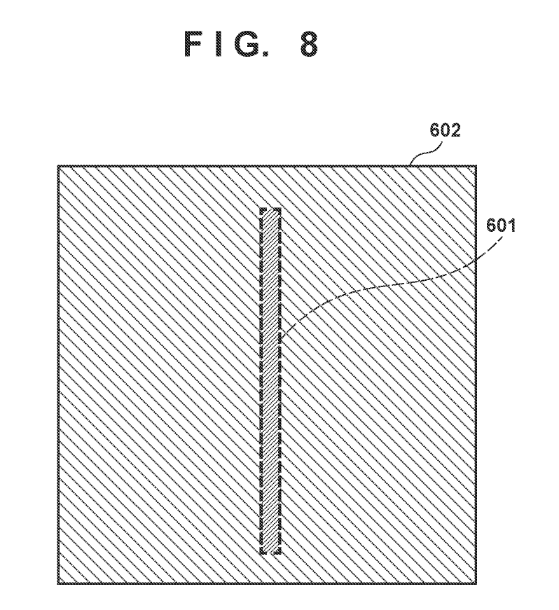

FIG. 8 is a diagram illustrating a two-dimensional image based on the average photon counts <n₁> and <n₂> obtained by applying the processing of the present embodiment. Although the substance 1 (601) and the substance 2 (602) cannot be distinguished between in the integral image of the radiation energy shown in FIG. 6, as stated in the description of FIG. 7, if the processing of the present embodiment is applied to obtain the average photon counts <n₁> and <n₂> corresponding to the multiple average energies E₁ and E₂ of the radiation and <n₂>/<n₁> is displayed, it is possible to make a distinction such that the substance 1 (601) has a larger value and the substance 2 (602) has a smaller value as shown in FIG. 8, for example.

According to the present embodiment, measurement in which the tube voltage is changed is not performed, the energy of radiation that is irradiated at a predetermined tube voltage can be approximated using multiple average energies, and the average photon counts corresponding to the multiple average energies can be calculated. In the present embodiment, as an example of the processing, an example was shown in which the average photon counts are calculated, but the total photon count that is not divided by the number of multiple instances of measurement m (m: an integer that is 2 or more) may be calculated.

Also, in the present embodiment, as the processing of the units of the data processing unit 106, an example was given in which each detection unit (detection element) performs processing, but it is also possible to collectively process the measurement information of multiple detection units (detection elements) for which average energies and average photon counts that are approximately the same can be expected. For example, in the multiple instances of measurement processing (step S201), it is sufficient to perform processing for obtaining a sum by adding up the measurement information of the multiple detection units (detection elements) for which average energies and average photon counts that are about the same can be expected, as the target of obtaining the sum in Equation 2. The detection units (detection elements) for which average energies and average photon counts that are approximately the same can be expected are detection units that are arranged near each other among the multiple detection units (detection elements) that are arranged in a two-dimensional shape, for example. It is sufficient that the control unit 105 performs processing for comparing the measurement information of a detection unit of interest and multiple peripheral detection units arranged in the periphery of the detection unit of interest and obtaining a sum by adding up the measurement information using the detection units for which the result of the comparison is within a predetermined threshold. The configuration of the present embodiment can also be used in a configuration in which dual energy imaging is performed on a subject using two types of radiation with different energies, and it is possible to further increase the average energy count by using the configuration of the present embodiment in the configuration for dual energy imaging as well.

### Second Embodiment

In the second embodiment, processing for determining the average energy based on an evaluation index in the processing for determining the average energy will be described. In the present embodiment, the configuration of the radiation imaging apparatus and the flow of the calculation processing for the average photon count are almost the same as in the first embodiment, but the processing content (step S205) executed by the average energy determination unit 108 differs.

With the present embodiment, in the average energy determination processing (step S205), the average energy is determined such that the value of an evaluation index f is optimized. Equation 10 illustrates an evaluation index f (evaluation function) that is used by the average energy determination unit 108 for average energy determination processing of the second embodiment.

$$f = \frac{1}{2} {\sum }_{ξ} {\{ 〈 {n}_{1} 〉 ( ξ ) - 〈 {n}_{2} 〉 ( ξ ) \}}^{2} \qquad Equation 10$$

The average energy determination unit 108 performs calculation processing for solving the optimization problem in which the average energies (E₁ and E₂) at which the value of the evaluation index f (evaluation function) reaches its maximum are obtained using the average energies E₁ and E₂ as variables. As the solution method for the optimization problem, the average energy determination unit 108 can execute calculation processing in which a Nelder-Mead method is applied, for example, and the average energy determination unit 108 can use the value obtained through the average energy determination processing (step S205) described in the first embodiment, for example, as the initial value of the solution method for solving the optimization problem. The solution method for the optimization problem is not limited to the Nelder-Mead method, and it is possible to perform calculation processing for solving the optimization problem for the evaluation index f (evaluation function) using another solution method.

Note that with the evaluation index f, <n₁>(ξ) is the average photon count corresponding to the average energy E₁ obtained by the ξ-th detection unit (detection element) of the radiation detection apparatus 104 (e.g., FPD). With the evaluation index f, the sum of Equation 8 obtains the sum of all of the detection units (detection elements) constituting the radiation detection apparatus 104. In the calculation processing for solving the optimization problem, calculation of the evaluation index f using the average photon count at the time of estimating the average energies E₁ and E₂ is needed, but it is sufficient to execute the average photon count calculation processing (step S206) for that. According to the present embodiment, an evaluation index is introduced and the average energies are determined such that the value of the evaluation index f is favorable, or in other words, such that the value of the evaluation index f is optimized.

### Third Embodiment

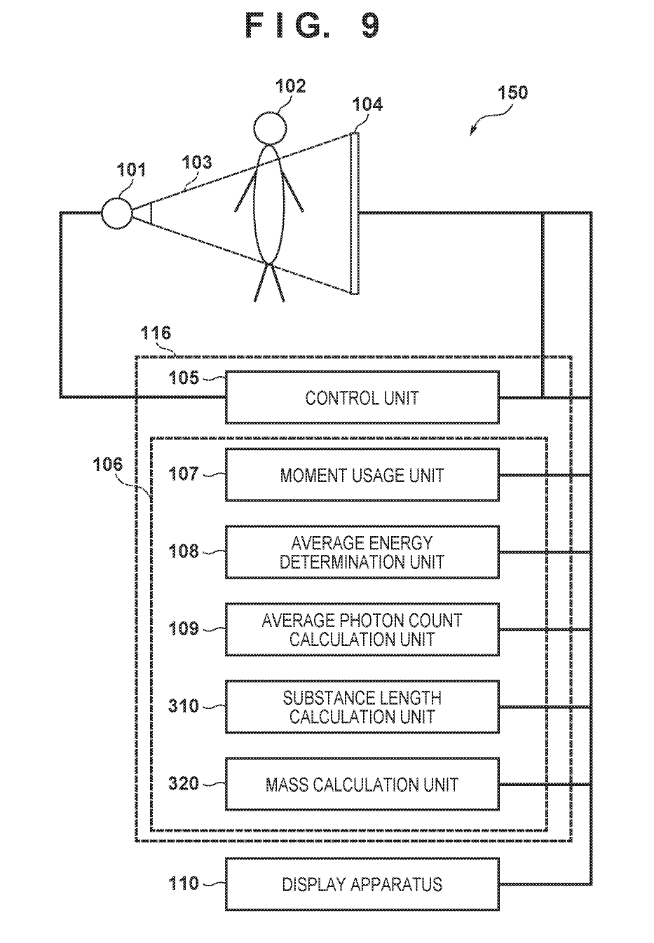

In the third embodiment, processing in which the average photon counts corresponding to the multiple energies are used to obtain the lengths of the substances constituting the object and processing in which the average photon counts corresponding to the multiple energies are used to obtain the masses per unit area of the substances constituting the object will be described. FIG. 9 is a diagram showing an example of a configuration of a radiation imaging apparatus 150 of the third embodiment. As shown in FIG. 9, the radiation imaging apparatus 150 includes a radiation generating apparatus 101, a radiation detection apparatus 104, and an information processing apparatus 116. Although the basic configuration is similar to that of the radiation imaging apparatus 100 shown in FIG. 1, in the present embodiment, the configuration of the data processing unit 106 of the information processing apparatus 116 differs from the functional configuration of the radiation imaging apparatus 100 described in FIG. 1 in that a substance length calculation unit 310 and a mass calculation unit 320 are included. The functions of the units of the substance length calculation unit 310 and the mass calculation unit 320 are configured using a program that is read from a CPU, a GPU, or a memory (not shown), for example. First, processing for obtaining the lengths of the substances constituting the object will be described. The substance length calculation unit 310 can calculate the lengths of the substances using the photon counts (average photon counts) calculated by the average photon count calculation unit 109, and the linear attenuation coefficients of the substances constituting the object. When the spectral distribution of the radiation irradiated from the radiation generating apparatus 101 is approximated using the multiple average energies as illustrated in the first embodiment, the average photon count <nⱼ> corresponding to the j-th average energy Eⱼ can be calculated using Equation 11.

[symbol]

nⱼ [symbol] = [symbol] sⱼ [symbol] exp(−∫μ(l,Eⱼ)dl)

Here, <sⱼ> is an average photon count having the average energy Eⱼ of the radiation irradiated from the radiation generating apparatus 101 to the detection unit (detection element) of the radiation detection apparatus 104, and μ(l, Eⱼ) is a linear attenuation coefficient of a position 1 corresponding to the average energy Eⱼ. Integration is performed on a linear path from the radiation generating apparatus 101 to the detection unit (detection element) of the radiation detection apparatus 104.

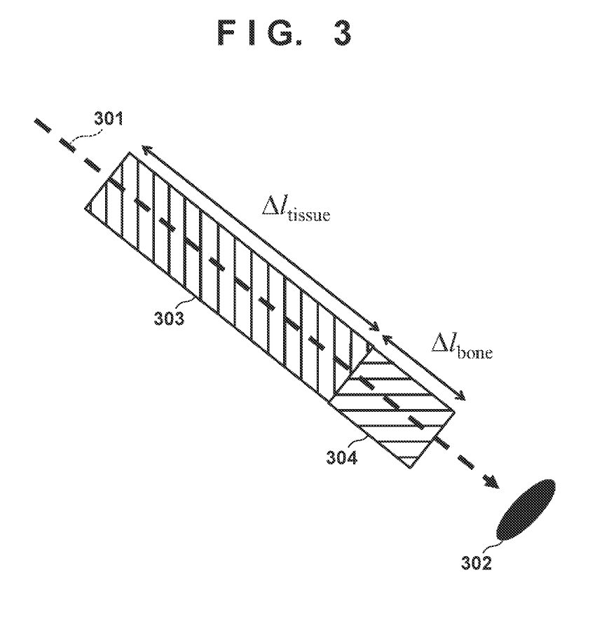

For example, in the case of FIG. 3, the radiation irradiated from the radiation generating apparatus 101 travels along a path 301 indicated by the broken-line arrow, is attenuated by passing through a substance 303 and a substance 304 included in the object, and is incident on the detection unit (detection element) 302. For example, if the substance 303 is soft tissue and the substance 304 is bone, Equation 11 can be written as Equation 12.

$$\begin{matrix}〈 {p}_{j} 〉 = {μ}_{tissue} ( {E}_{j} ) Δ {l}_{tissue} + {μ}_{bone} ( {E}_{j} ) Δ {l}_{bone} & Equation 12 \\ 〈 {p}_{j} 〉 = - log ( \frac{〈 {n}_{j} 〉}{〈 {s}_{j} 〉} ) & Equation 13\end{matrix}$$

In Equation 12, <pⱼ> is defined by Equation 13. μₜᵢₛₛᵤₑ(Eⱼ) is a linear attenuation coefficient of the soft tissue at the energy Eⱼ, and μ_{bone}(Eⱼ) is the linear attenuation coefficient of the bone at the energy Eⱼ. Δlₜᵢₛₛᵤₑ and Δl_{bone} are the length of the soft tissue and the length of the bone respectively. In the first embodiment, there were two types of average energies, namely E₁ and E₂, and therefore Equation 12 is two equations for the average energies E₁ and E₂. <sⱼ> can be obtained from the measurement result in the case of performing measurement with no object, and from the spectral distribution of the radiation irradiated from the radiation generating apparatus. <nⱼ> can be obtained using the calculation result of the average photon count calculation unit 109. Also, the linear attenuation coefficients μₜᵢₛₛᵤₑ(Eⱼ) and μ_{bone}(Eⱼ) can be obtained if the average density is inferred. The two variables Δlₜᵢₛₛᵤₑ and Δl_{bone} are undefined in Equation 12, and the lengths of the substances constituting the object can be obtained by solving the simultaneous linear equations using the two equations.

In the present embodiment, an example of a time at which there are two types of substances constituting the object was shown, but the gist of the present invention is not limited to this example. For example, in general, if the average photon counts corresponding to k types (k: an integer that is 2 or more) of average energies are obtained, the lengths of the k types of substances can be obtained. This is because the lengths of the k types of substance have k variables and the number of equations for the k types of average energies is k, and therefore by solving the simultaneous linear equations using K formulas, it is possible to obtain the lengths of the substances constituting the object. Note that in the case of obtaining the lengths of k′ (k′<k) types of substances, it is sufficient to use a minimum squares method or to reduce the linearly dependent formulas among the k formulas.

Next, processing for obtaining the masses per unit area of the substances constituting the object will be described. The mass calculation unit 320 can calculate the masses per unit area of the substances using the photon counts (average photon counts) calculated by the average photon count calculation unit 109 and the mass attenuation coefficients of the substances constituting the object. If the substances constituting the object are two types of substances, such as soft tissue and bone, the integration of Equation 11 can be written as Equation 14 using the mass attenuation coefficients.

∫μ(l,Eⱼ)dl=ϕₜᵢₛₛᵤₑ(Eⱼ)∫ρₜᵢₛₛᵤₑ(l)dl+ϕ_{bone}(Eⱼ)∫ρ_{bone}(l)dl

Here, φₜᵢₛₛᵤₑ is the mass attenuation coefficient of the soft tissue, φ_{bone} is the mass attenuation coefficient of the bone, ρᵢₛₛᵤₑ is the density of the soft tissue, and ρ_{bone} is the density of the bone. Equation 11 can be written as Equation 15 using these parameters.

[symbol]

pⱼ [symbol] =ϕₜᵢₛₛᵤₑ(Eⱼ)σₜᵢₛₛᵤₑ+ϕ_{bone}(Eⱼ)σ_{bone} Equation 15

σₜᵢₛₛᵤₑ=∫ρₜᵢₛₛᵤₑ(l)dl,σ_{bone}=∫ρ_{bone}(l)dl Equation 16

In Equation 15, σₜᵢₛₛᵤₑ and σ_{bone} are defined in Equation 16 and correspond to the masses per unit area. If there are two types of average energies, Equation 15 is two formulas, and therefore two variables (σₜᵢₛₛᵤₑ and σ_{bone}) can be obtained by solving the simultaneous linear equation using two formulas. By solving the simultaneous linear equation based on Equation 15, the masses per unit area of the substances constituting the object can be obtained.

In the present embodiment, an example of a time at which there are two types of substances constituting the object was shown, but the gist of the present invention is not limited to this example. For example, in general, if k types of average energies are obtained, similarly to the case of obtaining the lengths of the substances, the masses per unit area of the k types of substances can be obtained.

The control unit 105 can display the masses per unit area of the substances on the display unit 110. Also, the control unit 105 can display the lengths of the substances on the display unit 110. The control unit 105 can also display information indicating the lengths of the substances or the masses per unit area of the substances on the display apparatus 110 as diagnostic images by combining the images based on the average photon counts <n₁> and <n₂> described in the first embodiment.

According to the present embodiment, measurement in which the tube voltage is changed is not performed, and the lengths or masses per unit area of substances constituting an object can be obtained based on multiple average energies obtained by approximating the energies of the radiation irradiated at a predetermined tube voltage, or based on the average photon counts corresponding to the multiple average energies.

### Fourth Embodiment

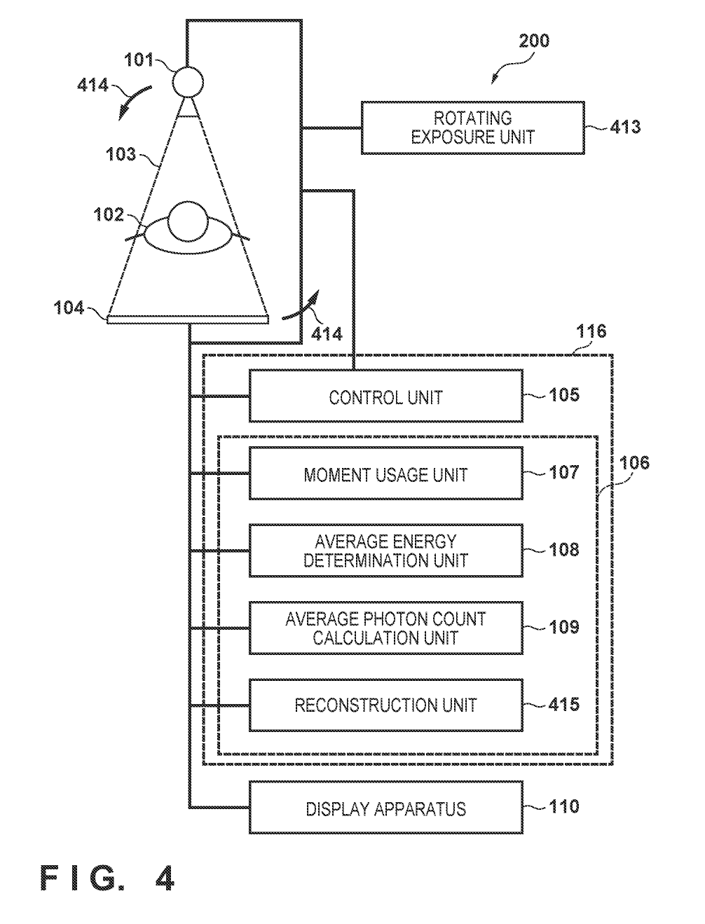

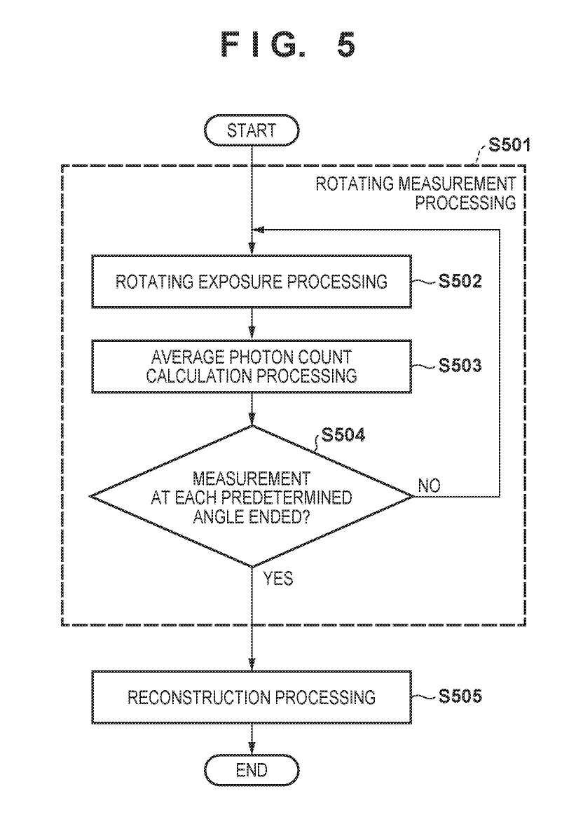

A fourth embodiment of the present invention will be described with reference to FIGS. 4 and 5. FIG. 4 is a diagram illustrating an apparatus configuration in the case of applying the present invention to a CT apparatus 200 and FIG. 5 is a diagram showing a processing flow for reconstructing linear attenuation coefficients for each average energy.

The apparatus configuration of the CT apparatus 200 shown in FIG. 4 differs from the apparatus configuration described using FIG. 1 in that a rotating exposure unit 413 and a reconstruction unit 415 are added. Note that the functions of the reconstruction unit 415 are configured using a program read from a CPU, a GPU, or a memory (not shown). Hereinafter, the differing apparatus configuration will be described. The apparatus configuration that is redundant with FIG. 1 will be omitted.

The rotating exposure unit 413 is a drive unit that synchronizes the radiation generating apparatus 101 and the radiation detection apparatus 104 and then performs driving so as rotate centered about the object 102, based on the control performed by the control unit 105. Arrow 414 indicates the rotation direction. Note that the rotation center need not be centered about the object 102, but rotation needs to be performed in a state in which the radiation generating apparatus 101 and the radiation detection apparatus 104 oppose each other on opposite sides of the object 102. In FIG. 4, the arrow 414 indicating the rotation direction is the rotation direction about a slice cross-section with respect to the object 102, but this example is not limited thereto, and for example, the object 102 may be scanned while the radiation generating apparatus 101 and the radiation detection apparatus 104 rotate in a direction orthogonal to the page surface of FIG. 4.

The reconstruction unit 415 can perform filter processing, back projection processing, and the like, and can perform reconstruction processing. In the present embodiment, the reconstruction unit 415 reconstructs the linear attenuation coefficients μ (E₁) and μ (E₂) corresponding to the multiple energies (average energies E₁ and E₂) based on the photon counts (average photon counts) corresponding to the multiple energies (average energies E₁ and E₂) obtained by the radiation imaging apparatus. The reconstruction unit 415 can perform, as a method for image reconstruction, a sequential approximation reconstruction method or an analytical reconstruction method, that is, reconstruction processing by unit of filtered back projection (FBP). For example, the functions of the reconstruction unit 415 are configured using a program read from a CPU, a GPU, or a memory (not shown). The configuration of the reconstruction unit 415 may be constituted by an integrated circuit or the like, as long as similar functions are achieved. For example, the reconstruction unit 415 performs filter processing on the measurement information output from the radiation detection apparatus 104, performs back projection processing or the like on the data obtained through the filter processing, and thus can reconstruct the multiple pieces of image data. When the back projection is complete and the reconstruction data is generated, the control unit 105 displays the generated reconstruction data and the like on the display apparatus 110.

FIG. 5 is a diagram illustrating a flow of operations of a CT apparatus. The operations performed by the apparatus configuration shown in FIG. 4 to calculate the linear attenuation coefficients for each average energy will be described with reference to FIG. 5.

### Rotating Measurement Processing

First, in step S501, the control unit 105 executes rotating measurement processing. The rotating measurement processing has three steps (steps S502 to S504). In step S502, the control unit 105 controls the rotating exposure unit 413 to rotate the radiation generating apparatus 101 and the radiation detection apparatus 104 centered about the object 102 to a predetermined rotation angle, and cause radiation to be emitted from the radiation generating apparatus 101. The control unit 105 controls the radiation generating apparatus 101 so as to irradiate radiation at a constant tube voltage, and causes the detection results (measurement information) of the radiation incident on the detection units (detection elements) of the radiation detection apparatus 104 to be output each certain period.

Next, in step S503, the average photon count calculation processing is executed. The processing of the present step corresponds to all of the steps (S201 to S206) of the flowchart described using FIG. 2. That is, in step S503, the multiple instances of measurement processing (step S201), the moment usage processing (step S204), the average energy determination processing (step S205), and the average photon count calculation processing (step S206) are executed, and the average photon counts (<n₁> and <n₂>) corresponding to the average energies (E₁ and E₂) of the radiation are calculated.

In step S504, the control unit 105 determines whether or not measurement at each predetermined angle has ended. If the measurement at each predetermined angle has not ended (step S504—No), the processing is returned to step S502 and the rotating exposure processing is executed. The control unit 105 controls the rotating exposure unit 413 to rotate the radiation generating apparatus 101 and the radiation detection apparatus 104 from the current rotation angle up to a further predetermined rotation angle, and causes the radiation to be emitted from the radiation generating apparatus 101.

On the other hand, if it is determined in step S504 that the measurement at each predetermined angle has ended (step S504—Yes), the processing is advanced to step S505. Note that the rotation angles at which imaging is executed can be set as appropriate. For example, angles obtained by evenly dividing a turn of 360° can be set as the predetermined angles. Also, in the present embodiment, the rotation angle of the radiation generating apparatus 101 and the radiation detection apparatus 104 is held in a state of having been rotated to a certain rotation angle, and thereafter the multiple instances of measurement processing are executed, but the gist of the present invention is not limited to this example. For example, it is also possible to use a method in which the radiation detection apparatus 104 performs multiple instances of measurement while the radiation generating apparatus 101 and the radiation detection apparatus 104 are rotated, and thereafter, the measurement information measured at adjacent rotation angles is collectively output, and moment usage processing is executed.

### Reconstruction Processing: Step S505

In step S505, the reconstruction unit 415 uses the average photon counts (<n₁> and <n₂>) for each of the average energies E₁ and E₂ to reconstruct the linear attenuation coefficients μ(E₁) and μ(E₂) corresponding to the average energies E₁ and E₂. The average photon counts obtained in step S503 before are used as the average photon counts (<n₁> and <n₂>). Also, as a method for reconstruction, the reconstruction unit 415 can obtain the linear attenuation coefficient μ(E₁) based on the average photon count <n₁> and can obtain the linear attenuation coefficient (E₂) based on the average photon count <n₂>, using a sequential approximation reconstruction method, a filter back projection (FBP) method, or the like, for example.

The control unit 105 can function as a display control unit to display, on the display apparatus 110, the linear attenuation coefficients μ(E₁) and μ(E₂) corresponding to the average energies E₁ and E₂ obtained through the reconstruction processing performed by the reconstruction unit 415, as diagnostic information. Also, the control unit 105 can combine the linear attenuation coefficients μ(E₁) and μ(E₂) corresponding to the average energies E₁ and E₂ with an image based on the average photon counts <n₁> and <n₂> described in the first embodiment and display them on the display apparatus 110 as diagnostic information.

According to the present embodiment, measurement in which the tube voltage is changed is not performed, linear attenuation coefficients of substances constituting an object corresponding to the average energies E₁ and E₂ can be obtained based on multiple average energies obtained by approximating the energies of the radiation irradiated at a predetermined tube voltage, and based on the average photon counts corresponding to the multiple average energies.

### Fifth Embodiment

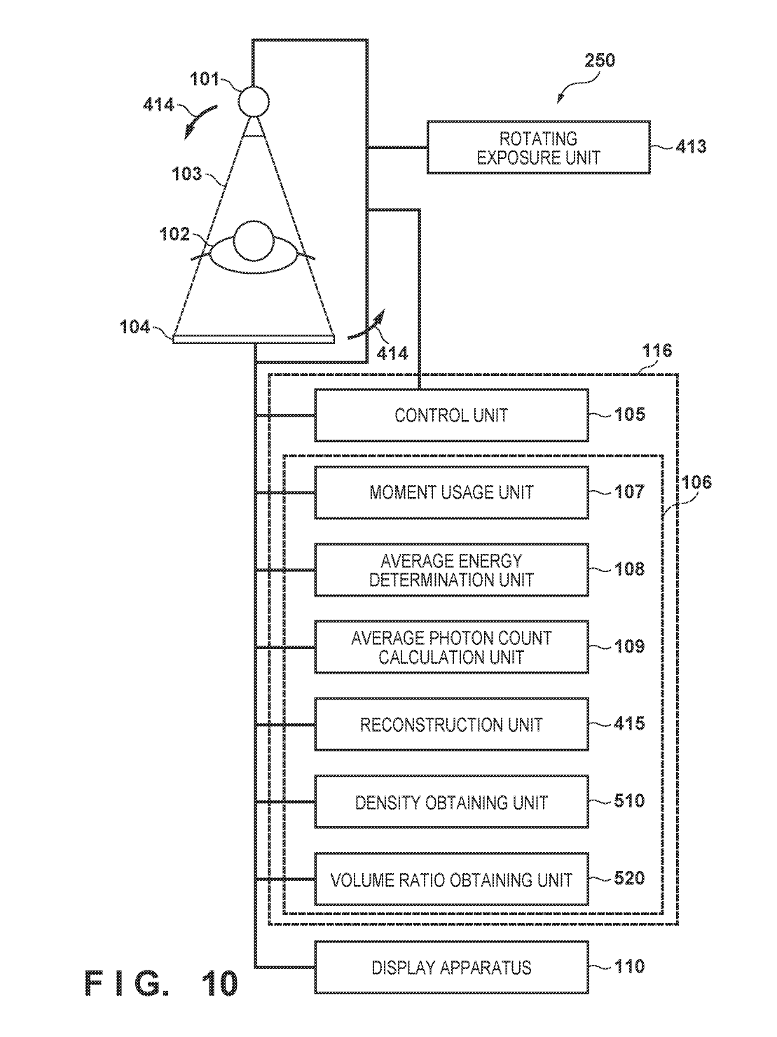

In the fifth embodiment of the present embodiment, processing for obtaining the densities of substances constituting an object and processing for obtaining volume ratios of the substances constituting the object using linear attenuation coefficients corresponding to multiple average energies will be described. FIG. 10 is a diagram showing a configuration example of a CT apparatus 250 according to the fifth embodiment. As shown in FIG. 10, the CT apparatus 250 includes a radiation generating apparatus 101, a radiation detection apparatus 104, a rotating exposure unit 413 that drives the radiation generating apparatus 101 and the radiation detection apparatus 104 so as to rotate in a state of opposing each other, and an information processing apparatus 116. Although the basic configuration is similar to that of the CT apparatus 200 shown in FIG. 4, in the present embodiment, the configuration of the data processing unit 106 of the information processing apparatus 116 differs from the functional configuration of the CT apparatus 200 illustrated in FIG. 4 in that a density obtaining unit 510 and a volume ratio obtaining unit 520 are included. The functions of the units of the density obtaining unit 510 and the volume ratio obtaining unit 520 are configured using a program that is read from a CPU, a GPU, or a memory (not shown), for example.

The density obtaining unit 510 can obtain the densities of the substances constituting the object based on the linear attenuation coefficients reconstructed by the reconstruction unit 415 and the mass attenuation coefficients of the substances constituting the object. Also, the volume ratio obtaining unit 520 can obtain the volume ratios of the substances constituting the object based on the linear attenuation coefficients reconstructed by the reconstruction unit 415 and the linear attenuation coefficients of the substances constituting the object. The linear attenuation coefficient μ(r, Eⱼ) at the position r inside of the object and energy Eⱼ can be written as Equation 17 using the mass attenuation coefficient. If multiple types of substances constituting an object are set via an input unit (not shown), the density obtaining unit 510 and the volume ratio obtaining unit 520 can obtain information on the mass attenuation coefficients and the linear attenuation coefficients corresponding to the multiple types of substances set via the input unit, and the information on the obtained mass attenuation coefficients and the linear attenuation coefficients can be used for calculation for obtaining the densities and the mass ratios of the substances.

$$μ ( r , {E}_{j} ) = {\sum }_{k = 1}^{{n}_{k}} {ρ}_{k} ( r ) {φ}_{k} ( {E}_{j} ) \qquad Equation 17$$

Here, nₖ is the number of types of substances constituting the object, φₖ is the mass attenuation coefficient of the k-th substance, and ρₖ is the density of the k-th substance. Equation 17 is a simultaneous linear equation in which the number of variables is nₖ, and if there are nₖ types of average energies of the radiation and the rank of the coefficient matrix of the simultaneous linear equation has not fallen, it is possible to solve the simultaneous linear equation and the solutions for the nₖ variables can be obtained. For example, since the linear attenuation coefficients corresponding to two types of average energies were obtained in the example shown in the fourth embodiment, if the substances constituting the object are of two types, namely soft tissue and bone, the densities of the substances (soft tissue and bone) can be obtained by solving the simultaneous linear equation.

Also, the linear attenuation coefficients can be written as Equation 18 using the volume ratios.

$$μ ( r , {E}_{j} ) = {\sum }_{k = 1}^{{n}_{k}} {c}_{k} {μ}_{k} ( r , {E}_{j} ) \qquad Equation 18$$

Here, cₖ is the volume ratio of the k-th substance. Also, μₖ is the linear attenuation coefficient of the k-th substance and can be obtained in advance if the density of the substance is inferred to be the average value. Equation 18 is a simultaneous linear equation in which the number of variables is nₖ, and if there are nₖ types of average energies of the radiation and the rank of the coefficient matrix of the simultaneous linear equation has not fallen, it is possible to solve the simultaneous linear equation and the solutions for the nₖ variables can be obtained. For example, since the linear attenuation coefficients corresponding to two types of average energies were obtained in the example shown in the fourth embodiment, if the substances constituting the object are of two types, namely soft tissue and bone, the mass ratios of the substances (soft tissue and bone) can be obtained by solving the simultaneous linear equation.

The control unit 105 displays, on the display apparatus 110, the processing results of the density obtaining unit 510 and the volume ratio obtaining unit 520. The control unit 105 can display the densities of the substances constituting the object or the volume ratios of the substances on the display apparatus 110. Also, the control unit 105 combines the information indicating the densities or the volume ratios of the substances constituting the object with an image based on the average photon counts <n₁> and <n₂> described in the first embodiment and displays the result as a diagnostic image on the display apparatus 110.

According to the present embodiment, measurement in which the tube voltage is changed is not performed, and the densities or volume ratios of substances constituting an object can be obtained based on multiple average energies obtained by approximating the energies of the radiation irradiated at a predetermined tube voltage.

According to the present invention, it is possible to obtain multiple pieces of energy information of radiation that is irradiated based on a constant tube voltage, and to calculate the photon counts corresponding to the pieces of energy information with high precision, without being influenced by a decrease in the measurement accuracy. That is, according to the present invention, it is possible to calculate the photon counts with high precision while reducing the burden on the operator, without requiring switching of the tube voltage.

Also, according to the present invention, it is possible to generate an image of an object including substances that cannot be discriminated between with only a radiation energy image, by using a conventional radiation detection apparatus to image the photon counts of radiation having different energies.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A radiation imaging apparatus comprising:
a detection unit configured to obtain measurement information based on a detection result of radiation irradiated based on a constant tube voltage;
an obtaining unit configured to obtain second measurement information of the radiation based on a moment of the measurement information obtained by detecting the radiation a plurality of times;
an energy determination unit configured to determine a plurality of energies for approximating an energy distribution of the radiation; and
a calculation unit configured to calculate photon counts corresponding to the plurality of energies based on the second measurement information.

2. A radiation imaging apparatus comprising:
a detection unit configured to obtain measurement information based on a detection result of radiation irradiated based on a constant tube voltage;
an obtaining unit configured to obtain second measurement information of the radiation based on a moment of the measurement information obtained by detecting the radiation a plurality of times;
an energy determination unit configured to determine a plurality of energies for approximating an energy distribution of the radiation;
a calculation unit configured to calculate photon counts corresponding to the plurality of energies based on the second measurement information; and
a display control unit configured to display an image based on the photon counts on a display unit.

3. The radiation imaging apparatus according to claim 1, wherein based on the moment of the measurement information, the obtaining unit obtains, as the second measurement information, an average photon count of the radiation incident on the detection unit.

4. The radiation imaging apparatus according to claim 1, wherein the calculation unit calculates the photon counts corresponding to the plurality of energies using the plurality of energies, an average value of the measurement information, and the second information.

5. The radiation imaging apparatus according to claim 1, wherein the energy determination unit divides a spectral distribution of the radiation into a plurality of regions and determines average values of energy based on spectral distributions in the divided regions as energies for approximating the energy distribution of the radiation.

6. The radiation imaging apparatus according to claim 5, wherein the energy determination unit divides the spectral distribution of the radiation into a plurality of regions based on energies of absorption edges of substances constituting an object.

7. The radiation imaging apparatus according to claim 2, further comprising a substance length calculation unit configured to calculate lengths of substances constituting an object, using the photon counts and linear attenuation coefficients of the substances.

8. The radiation imaging apparatus according to claim 2, further comprising a mass calculation unit configured to calculate per unit area of substances constituting an object, using the photon counts and mass attenuation coefficients of the substances.

9. The radiation imaging apparatus according to claim 7, wherein the display control unit displays the masses per unit area of the substances on the display unit.

10. The radiation imaging apparatus according to claim 8, wherein the display control unit displays the lengths of the substances on the display unit.

11. The radiation imaging apparatus according to claim 1, further comprising
an imaging control unit configured to control operations of a radiation generating unit configured to irradiate radiation and the detection unit,
wherein the imaging control unit causes the radiation generating unit to irradiate the radiation based on a constant tube voltage, and
the imaging control unit controls the detection unit to output a detection result of the radiation incident on the detection unit each certain period and obtain the measurement information.

12. A CT apparatus comprising:
the radiation imaging apparatus according to claim 1; and
a reconstruction unit configured to reconstruct the linear attenuation coefficients corresponding to the plurality of energies obtained by the radiation imaging apparatus, based on photon counts corresponding to the plurality of energies.

13. The CT apparatus according to claim 12, further comprising a density obtaining unit configured to obtain densities of the substances constituting the object based on the reconstructed linear attenuation coefficients and the mass attenuation coefficients of the substances.

14. The CT apparatus according to claim 13, further comprising a volume ratio obtaining unit configured to obtain volume ratios of the substances constituting the object based on the reconstructed linear attenuation coefficients and the linear attenuation coefficients of the substances.

15. The CT apparatus according to claim 14, further comprising
a display control unit configured to display a processing result of the reconstruction unit on the display unit,
wherein the display control unit displays, on the display unit, the densities of the substances or the volume ratios of the substances.

16. A radiation imaging method comprising:
a step of detecting, with a detection unit, radiation irradiated based on a constant tube voltage;
a step of obtaining second measurement information of the radiation based on a moment of the measurement information obtained by detecting the radiation a plurality of times;
a step of determining a plurality of energies for approximating an energy distribution of the radiation; and
a step of calculating photon counts corresponding to the plurality of energies based on the measurement information and the second measurement information.

17. A radiation imaging method comprising:
a step of detecting, with a detection unit, radiation irradiated based on a constant tube voltage;
a step of obtaining second measurement information of the radiation based on a moment of the measurement information obtained by detecting the radiation a plurality of times;
a step of determining a plurality of energies for approximating an energy distribution of the radiation;
a step of calculating photon counts corresponding to the plurality of energies based on the measurement information and the second measurement information; and
a step of displaying an image based on the photon counts on a display unit.

18. A non-transitory computer-readable storage medium storing a program for causing a computer to execute the steps of the radiation imaging method according to claim 16.
